(19) United States
(12) Patent Application Publication
(10) Pub. No.: US 2026/0167913 A1
(43) Pub. Date: Jun. 18, 2026

(54) CELL CULTURE DEVICE AND MULTI-REGION LIQUID PATTERNING METHOD USING THE SAME

(71) Applicant: Seokyoung Bang, Seoul (KR)

(72) Inventors: Seokyoung BANG, Seoul (KR); Seonghun Mun, Chuncheon-si (KR); Minkyeong Jeong, Gongju-si (KR); Dohyung Kim, Seoul (KR)

(73) Assignee: Seokyoung Bang, Seoul (KR)

(21) Appl. No.: 19/375,063

(22) Filed: Oct. 30, 2025

(30) Foreign Application Priority Data
Oct. 30, 2024 (KR) 10-2024-0150465
Oct. 29, 2025 (KR) 10-2025-0159492

(51) Int. Cl. C12M 1/32; C12M 3/00

(52) U.S. Cl. CPC C12M 23/12; C12M 23/44

(57) ABSTRACT

The present invention provides a cell culture device including a culture structure portion including a plurality of cell culture regions, wherein, in the culture structure portion, a culture medium for culturing a cell is maintained in each of the plurality of cell culture regions due to surface tension, and the plurality of cell culture regions are formed with a through-type structure so that an interaction is possible between adjacent cell culture regions among the plurality of cell culture regions.

### CROSS-REFERENCE TO RELATED APPLICATION

This application claims priority to and the benefit of Korean Patent Application Nos. 10-2024-0150465 filed on Oct. 30, 2024 and 10-2025-0159492 filed on Oct. 29, 2025, the disclosure of which is incorporated herein by reference in its entirety.

### TECHNICAL FIELD

The present invention relates to a cell culture device and a liquid patterning technology for cell culture, and more particularly, to a cell culture device capable of simultaneously culturing various cells in multiple regions by performing liquid patterning in each of a plurality of cell culture regions.

### BACKGROUND ART

Cell culture technology forms the foundation of life science and medical research and has become an indispensable tool in various fields of application such as exploring the fundamental mechanisms of diseases, developing new drugs, advancing tissue engineering, and conducting toxicity assessments. In particular, cell culture plays a pivotal role in modeling complex biological systems and conducting in-depth research on cell-cell interactions in laboratories.

However, most existing cell culture methods have been conducted on a two-dimensional plane and thus have an intrinsic limitation of not being able to sufficiently reflect a complex three-dimensional environment in which cells exist in the natural world. These two-dimensional culture methods have limitations in accurately replicating cell-cell interactions and formation of three-dimensional structures of tissues, and there are concerns that such limitations may cause limitations in research reliability and practical applicability of results of the research. In addition, the existing methods have various limitations in simultaneously culturing various cell types and effectively observing interactions between them. In particular, in a cell co-culture system, there is a problem that it is difficult to set and maintain culture conditions optimized for different cell types, and there is a concern that such a problem may decrease cell culture efficiency and accuracy of experimental results.

Therefore, in this industry, there may be demand for research and development of a new cell culture device for overcoming the limitations of the conventional cell culture technologies and expanding the reliability and scope of application of cell research.

### PRIOR ART DOCUMENTS

### Patent Documents

Korean Patent Application Publication No. 10-2023-0042782 (Mar. 30, 2023)

### SUMMARY OF THE INVENTION

### Problems to be Solved

The present invention is directed to providing a three-dimensional cell culture device capable of simultaneously culturing various cell types and observing interactions therebetween while more accurately replicating a cell environment.

The objects of the present invention are not limited to the above-mentioned object, and other unmentioned objects will be able to be clearly understood by those skilled in the art from the following description.

### Means for Solving the Problems

According to a first embodiment of the present invention, there is provided a cell culture device including a culture structure portion including a plurality of cell culture regions, wherein, in the culture structure portion, a culture medium for culturing a cell is maintained in each of the plurality of cell culture regions due to surface tension, and the plurality of cell culture regions are formed with a through-type structure so that an interaction is possible between adjacent cell culture regions among the plurality of cell culture regions.

Preferably, the plurality of cell culture regions may each be formed with a through-type cube structure of a predetermined size, and a plurality of different cells may be simultaneously cultured through different culture media located in the through-type cube structures.

Preferably, based on an inner space of the through-type cube structure, the through-type cube structure may have a size in which each of a width, a length, and a height ranges from 0.35 mm to 2 mm.

Preferably, the culture structure portion may further include a coupling portion mechanically coupled to another cell culture device, and an interaction may be possible between cell culture regions of a plurality of cell culture devices coupled through the coupling portion.

Preferably, the coupling portion may include a plurality of through-holes to enable vertical coupling with the other cell culture device through a holder having a specific structure, and an interaction between cells may be possible due to contact between culture media of corresponding cell culture regions of the vertically-coupled plurality of cell culture devices.

Preferably, the coupling portion may include an embossed or engraved structure portion to enable horizontal coupling with the other cell culture device, and an interaction between cells may be possible due to contact between culture media of cell culture regions located on coupling surfaces of the horizontally-coupled plurality of cell culture devices.

Preferably, the culture structure portion may further include cylindrical portions formed at both ends of the plurality of cell culture regions to enable liquid perfusion to a series of cell culture regions among the plurality of cell culture regions. According to a second embodiment of the present invention, there is provided a multi-region liquid patterning method using a cell culture device including a culture structure portion including a plurality of cell culture regions, the multi-region liquid patterning method including: patterning a liquid inside each of the plurality of cell culture regions by passing a pipette containing a culture medium for cell culture through each of the plurality of cell culture regions and then, when lifting the pipette, using surface tension generated between the culture medium formed on a tip of the pipette and the cell culture regions formed with a through-type structure, wherein an interaction occurs between adjacent cell culture regions among the plurality of cell culture regions.

Preferably, the patterning of the liquid may include patterning a culture medium including human vascular endothelial cells inside each of the plurality of cell culture regions of the cell culture device, patterning a culture medium including human lung fibroblasts inside each of a plurality of cell culture regions of another cell culture device that is able to be vertically coupled to the cell culture device, mechanically coupling the cell culture device and the other cell culture device through a coupling portion formed in a culture structure portion so that the cell culture device is located below and the other cell culture device is located above, and forming a vascularized artificial tissue in the cell culture device located below according to an interaction between cells that occurs due to contact between culture media of corresponding cell culture regions of the cell culture device and the other cell culture device.

Preferably, the patterning of the liquid may include patterning a sol-state hydrogel inside a series of cell culture regions having cylindrical portions formed at both ends among the plurality of cell culture regions, passing a microneedle through the series of cell culture regions via the cylindrical portions and inducing gelation, and when the microneedle is removed after the gelation, forming a vascular structure in which liquid perfusion is possible.

Other details of the present disclosure are incorporated in the detailed description and the drawings.

### Advantageous Effects of the Invention

According to various embodiments of the present invention, by presenting a new methodology for simultaneously culturing different cell types, it is possible to provide an effect of significantly improving efficiency of cell co-culture and accuracy of experiments.

In addition, optimized culture conditions can be set by applying a customized culture medium suitable for each cell type, and in this way, several variables that may occur in a cell culture process can be controlled, and the growth of and interactions between cells can be more precisely observed.

In addition, through a flexible coupling structure between cell culture devices, interactions between various cell types are facilitated, and cell culture media can be mixed or exchanged as necessary. In this way, there are advantages of widening the range of cell culture experiments and assisting in multidimensional cell research.

Further, by providing a new way of reducing dependence on animal experiments and evaluating effects of drugs using a non-animal method, the present invention can contribute to promoting research that takes bioethics into consideration. The effects of the present invention are not limited to the above-mentioned effects, and other unmentioned effects should be clearly understood by those skilled in the art from the above description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects will be described with reference to the accompanying drawings, and here, like reference numerals are used to collectively designate like components. In the following embodiments, for the purpose of description, multiple detailed matters are presented to provide overall understanding of one or more aspects. However, it is obvious that the aspect(s) may be embodied without the detailed matters.

FIG. 1 is a view of a cell culture device according to a preferred embodiment of the present invention.

FIG. 2 is a view for describing a multi-region liquid patterning method using the cell culture device according to one embodiment.

FIGS. 3 to 5 are views for describing an interaction between cell culture regions of the cell culture device according to one embodiment.

FIG. 6 is a view showing an experimental result related to the interaction between the cell culture regions of the cell culture device according to one embodiment.

FIG. 7 is a view showing vertical coupling of a plurality of cell culture devices according to one embodiment.

FIGS. 8 and 9 are views for describing an interaction between cell culture regions according to the vertical coupling of the plurality of cell culture devices according to one embodiment.

FIG. 10 is a view showing horizontal coupling of a plurality of cell culture devices according to one embodiment.

FIG. 11 is a view for describing an interaction between cell culture regions according to the horizontal coupling of the plurality of cell culture devices according to one embodiment.

FIG. 12 is a view of a cell culture device including a cylindrical portion according to one embodiment.

FIG. 13 is a view for describing a vascular structure formed using the cell culture device including the cylindrical portion according to one embodiment.

FIGS. 14 and 15 are views for describing an interaction between cell culture regions using the vascular structure formed in the cell culture device including the cylindrical portion according to one embodiment.

### DETAILED DESCRIPTION OF THE INVENTION

Various embodiments and/or aspects will now be disclosed with reference to the accompanying drawings. In the following description, for the purpose of description, multiple detailed matters will be disclosed in order to help comprehensive appreciation of one or more aspects. However, those skilled in the art will recognize that the aspect(s) can be executed without the detailed matters. In the following disclosure and the accompanying drawings, specific exemplary aspects of one or more aspects will be described in detail. However, the aspects are exemplary and some of various methods in principles of various aspects may be used, and the descriptions are intended to include all of the aspects and equivalents thereof. Specifically, in “embodiment,” “example,” “aspect,” “illustration,” and the like used in the specification, it may not be construed that a predetermined aspect or design which is described is more excellent or advantageous than other aspects or designs.

Hereinafter, identical or similar components will be assigned the same reference numbers regardless of drawing symbols, and overlapping descriptions thereof will be omitted. In addition, in describing an embodiment disclosed in the present specification, when it is determined that detailed description of a related known technology may obscure the gist of the embodiment disclosed in the present specification, the detailed description thereof will be omitted. In addition, the accompanying drawings are only intended to make the embodiment disclosed in the present specification easier to understand, and the technical idea disclosed in the present specification is not limited by the accompanying drawings.

Although the terms “first,” “second,” etc., may be used herein to describe various elements or components, these elements or components should not be limited by these terms. These terms are only used to distinguish one element or component from another. Thus, a first element or component mentioned below may be a second element or component within the technical idea of the present invention.

Unless otherwise defined, all terms (including technical and scientific terms) used herein may be used with meanings that may be commonly understood by those of ordinary skill in the art to which the present invention pertains. Also, terms defined in commonly used dictionaries should not be interpreted in an idealized or overly formal sense unless expressly so defined herein.

In addition, the term “or” is intended to mean not exclusive “or” but inclusive “or.” That is, unless specified otherwise or clearly stated in the context, “X uses A or B” is intended to mean one of the natural inclusive substitutions. That is, “X uses A or B” may apply to any of the case where X uses A, the case where X uses B, or the case where X uses both A and B. Further, it should be understood that the term “and/or” used herein designates and includes all possible combinations of one or more items among listed related items.

Also, the terms “include” and/or “including” should be understood as indicating the presence of corresponding features and/or components but not excluding the presence or addition of one or more other features, components, and/or groups thereof. Also, unless specified otherwise or the context clearly indicates singularity, a singular expression should be generally interpreted as indicating “one or more” in the present specification and the claims.

When a certain component is mentioned as being “connected” or “linked” to another component, it should be understood that the component may be directly connected or linked to the other component, or another component may be present therebetween. On the other hand, when a certain component is mentioned as being “directly connected” or “directly linked” to another component, it should be understood that no other component is present between the two components.

When a component or layer is referred to as being “on” another component or layer, this includes not only a case in which the component or layer is right above the other component or layer but also a case in which another layer or component is interposed therebetween. On the other hand, when a component is referred to as being “directly on” or “right above” another component, this indicates that no other component or layer is interposed therebetween.

Spatially relative terms such as “below,” “beneath,” “lower,” “above,” and “upper” may be used to easily describe the relationship between one component and other components illustrated in the drawings. Spatially relative terms should be understood as encompassing different directions of an element that is in use or operating, in addition to directions depicted in the drawings.

Objects and effects of the present invention and technical configurations for achieving the same will become apparent with reference to embodiments described in detail below and the accompanying drawings. In describing the present invention, when detailed description of known functions or configurations is determined as having the possibility of unnecessarily obscuring the gist of the present invention, the detailed description thereof will be omitted. Also, the terms described below are terms defined in consideration of their functionality in the present invention and may vary depending on an intention of a user or an operator, customary practice, or the like.

However, the present invention is not limited by embodiments disclosed below and may be implemented in various different forms. The present embodiments are provided to make the present invention complete and to fully inform those of ordinary skill in the art to which the present invention pertains of the scope of the disclosure, and the present invention is only defined by the scope of the claims. Therefore, the definition should be made based on the entire content of the present specification.

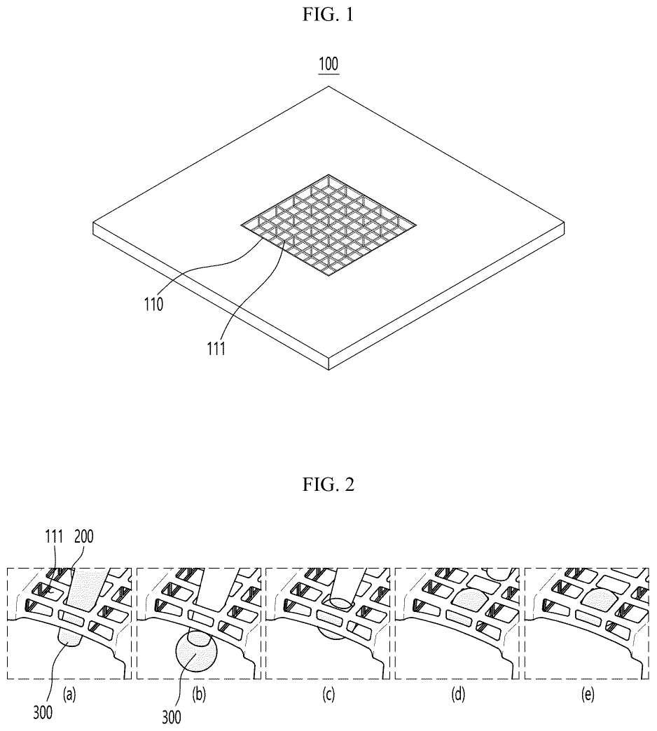

FIG. 1 is a view of a cell culture device according to a preferred embodiment of the present invention.

Referring to FIG. 1, a cell culture device 100 includes a culture structure portion 110, and the culture structure portion 110 includes a plurality of cell culture regions 111.

The cell culture device 100, which is a device for artificially culturing cells under predetermined conditions in a laboratory environment, artificially maintains an environment such as nutrition, temperature, or oxygen, to allow cell culture. Preferably, the cell culture device 100 may simultaneously culture various cells using a culture medium suitable for each cell. The cell culture device 100 may be manufactured using stereolithography (SLA) 3D printing, for example, may be manufactured by being printed using black resin V4.1 and then undergoing a post-processing process that corresponds to 10 minutes of washing and 30 minutes of UV curing at 60° C. Here, the type of resin used during 3D printing and the post-processing process may be changed in various ways. The cell culture device 100 manufactured in this way may include a structure related to the culture structure portion 110 and the plurality of cell culture regions 111.

The culture structure portion 110 is an entire region in which cells are cultured with culture media and is made of the plurality of cell culture regions 111. The plurality of cell culture regions 111 are regions in which culture media for culturing cells are maintained due to surface tension, and a plurality of different cells can be simultaneously cultured by different culture media each being patterned in one of the plurality of cell culture regions 111. Preferably, each of the plurality of cell culture regions 111 may be formed as a through-type cube structure of a predetermined size, and an interaction may occur between adjacent cell culture regions. Here, based on an inner space of a cube, the through-type cube structure may have a size in which each of a width, a length, and a height ranges from 0.35 mm to 2 mm, and based on a size also including an outer frame of a cube, the through-type cube structure may have a size in which each of a width, a length, and a height ranges from 0.65 mm to 2.3 mm, but the size of the through-type cube structure may be changed in various ways within a range in which surface tension of culture media is maintained. In addition, the sizes of the through-type cube structures of the plurality of cell culture regions 111 may be identically formed within the range satisfying the above conditions, the number of the plurality of cell culture regions 111 constituting the culture structure portion 110 or the number of culture structure portions 110 included in the cell culture device 100 may be changed in various ways, and the size of the cell culture device 100 may also be changed in various ways according to the purpose.

Preferably, the cell culture device 100 according to the present invention enables cell co-culture in which spatial and temporal control is possible because different cells can be cultured in each of the plurality of cell culture regions 111. In addition, although the cell culture device 100, the culture structure portion 110, and the plurality of cell culture regions 111 are each illustrated as having a quadrangular shape in FIG. 1, the shapes and sizes thereof may be changed in various ways.

FIG. 2 is a view for describing a multi-region liquid patterning method using the cell culture device according to one embodiment.

Referring to FIG. 2, liquid patterning may be performed in each of the plurality of cell culture regions 111 formed in the culture structure portion 110 of the cell culture device 100 through each step illustrated in (a) to (e).

When a pipette 300 containing a culture medium 200 for cell culture is passed through the cell culture region 111 as illustrated in (a) of FIG. 2, the culture medium 200 is formed on a tip of the pipette 300 as illustrated in (b), and the pipette 300 is lifted in a state in which the culture medium 200 is formed on the tip of the pipette 300 as illustrated in (c), surface tension is generated between the culture medium 200, which is a liquid or a hydrogel, and the cell culture region 111 as illustrated in (d), and the culture medium 200 is separated from the tip of the pipette 300 and patterned in the cell culture region 111 as illustrated in (e). In addition, after liquid patterning is performed in the plurality of cell culture regions 111, the culture medium 200 patterned in a specific cell culture region 111 may be removed by an aspiration method. That is, a pipette or a vacuum suction device may be placed in a specific cell culture region 111, in which liquid patterning has been performed, to remove only the culture medium 200 patterned in the specific cell culture region 111. In this way, patterning may be selectively performed only in a specific cell culture region 111 among the plurality of cell culture regions 111.

In various embodiments, such a patterning method may be performed using an automated patterning device. For example, the patterning method according to the above-described method may be performed using a patterning device at a position designated by a user among the plurality of cell culture regions 111 formed in the culture structure portion 110 of the cell culture device 100.

According to an embodiment, a user interface that allows the user to designate a patterning position among the plurality of cell culture regions 111 and allows patterning to be performed according thereto may be provided through a computing device.

Preferably, different culture media 200 may each be patterned in one of the plurality of cell culture regions 111 formed in the culture structure portion 110 of the cell culture device 100 according to the liquid patterning method illustrated in FIG. 2, and different cells may be cultured using the different culture media 200. The patterned culture media 200 and the cells types are not limited, and the same culture medium 200 may be patterned and the same cell may be cultured in the plurality of cell culture regions 111.

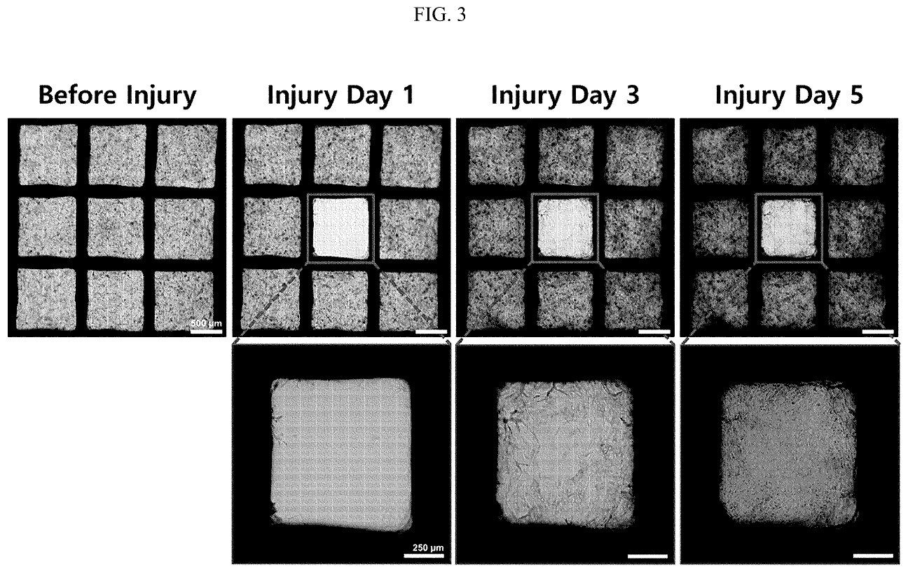

When the plurality of cell culture regions 111 are each formed to have the through-type cube structure, and different culture media 200 are each patterned in one of the plurality of cell culture regions 111, an interaction is possible between the culture media 200 patterned in adjacent cell culture regions 111. An interaction between the culture media 200 will be described in more detail below with reference to FIGS. 3 to 6.

Referring to FIG. 3 which is an exemplary view of an observed state in which cells are simultaneously cultured in the plurality of cell culture regions 111, first, a liquid-state suspension of 3T3 fibroblasts and collagen type I gel (for example, the concentration of collagen type I gel corresponds to 2.5 mg/ml) was patterned in the cell culture device 100, gelled, and then cultured for 5 days to allow a tissue to be stabilized in the cell culture regions 111 as illustrated in “Before Injury.” Then, a hydrogel patterned in a central region (center voxel) (the region marked with a red box in FIG. 3) among the cell culture regions 111 was removed by an aspiration method to induce an injury, the central region was filled again with a fibrin gel not containing a cell, and culturing was performed to replicate thrombus (fibrin network) generation at an early stage of an injury healing process as illustrated in “Injury Day 1.”

Preferably, prior to performing patterning in the cell culture device 100, cells may be proliferated through the following simple culture so that the number of cells becomes a number suitable for the corresponding experiment (for example, a suitable number of cells may be preset to a specific range according to an experiment). Mouse NIH/3T3 fibroblasts are cultured in Dulbecco's Modified Eagle Medium (DMEM), that is, D10, containing 10% (vol/vol) fetal bovine serum (FBS) and 1% (vol/vol) penicillin-streptomycin (P/S) antibiotic mixture, a passage number is 12 to 16, and a media change is carried out once every 2 to 3 days. For subculture, NIH/3T3 cells are detached by treating them with 0.25% trypsin-EDTA, which is an enzyme mixture, centrifuged at 169×g for 3 minutes, and resuspended in the corresponding cell culture medium. This cell culture may be continued until the cell count reaches a number in a specified range.

Preferably, the above-described replication of thrombus generation at an early stage of an injury healing process may be performed in the following manner using 3T3 fibroblasts. 3T3 fibroblasts are suspended in collagen type I gel to prepare a suspension with a gel concentration of 2.5 mg/ml and a cell concentration of 2.0×10⁶ cells/ml. 1.2 μl of the suspension is patterned in each of the plurality of cell culture regions 111 and then gelled for 30 minutes in an incubator in which a temperature is maintained at 37° C. and to which 5% CO₂ is supplied. After gelation, the cell culture device 100 is immersed in a structure filled with D10 to perform culture for 5 days, and a media change is carried out daily. After 5 days, the gel in the central region is removed by aspiration. The central region is filled with 1.2 μl of fibrin gel again, gelation is induced for 5 minutes at normal temperature, culture is performed again in the structure filled with D10, and a media change is carried out daily.

Here, the fibrin gel with which the central region is filled may be prepared through the following process. Bovine fibrinogen, a fibrinogen protein extracted from cows, is dissolved at a concentration of 10 mg/ml in Dulbecco's phosphate-buffered saline (DPBS) to prepare a fibrinogen solution, and to prevent fibrinolysis, aprotinin (0.15 U/ml; Sigma-Aldrich, USA) is mixed with the fibrinogen solution in a volume ratio of 25:4. A fibrinogen solution with a final concentration of 2.11 mg/ml is produced with this process, Thrombin (0.5 U/ml; Sigma-Aldrich, USA) is added in a volume ratio of 50:1 thereto, and the central region is filled again with 1.2 μl of the resulting product.

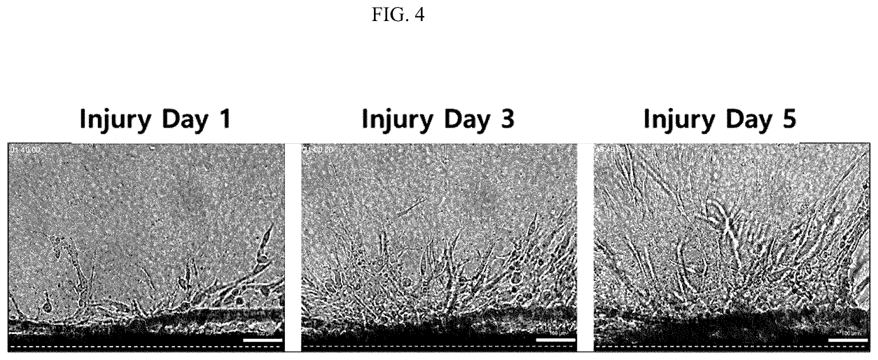

Referring to the central region of “Injury Day 1,” “Injury Day 3,” and “Injury Day 5” of FIG. 3, it can be seen that cells migrate and enter the central region from peripheral regions of the central region when additional culture is performed after an injury is induced, and it can be confirmed in detail through FIG. 4 that cells migrate to the central region from adjacent cell culture regions 111.

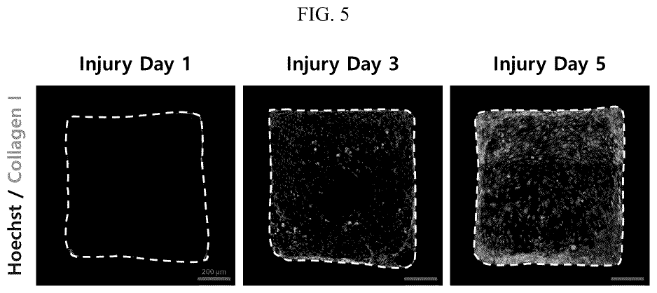

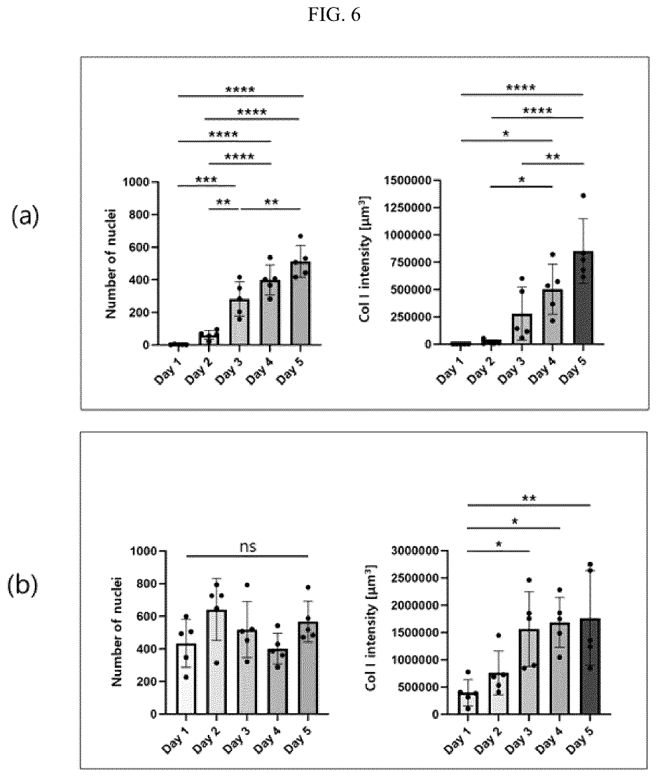

In addition, referring to FIG. 5 which is an exemplary view of confocal microscopic images of the central region by culture date, from changes by culture date of cells stained in blue and collagen type I stained in green, it can be confirmed that cells migrated from the adjacent cell culture regions 111 to the central region secrete collagen.

Preferably, immunofluorescence staining and imaging illustrated in FIG. 5 may be performed through the following process. To analyze the central region, the nuclei of cells located in the central region, and collagen type I secreted by the cells, immunofluorescence staining for collagen type I is performed using Hoechst 33342, a DNA staining fluorescent dye. A sample is washed three times with DPBS, a buffered saline solution, and fixed with 4% paraformaldehyde (PFA) for 30 minutes. Then, the sample is washed three times with DPBS and treated with a blocking solution, prepared by dissolving 1% (wt/vol) bovine serum albumin (BSA), 2% (vol/vol) fetal bovine serum (FBS), and 0.2% (vol/vol) nonionic surfactant (Triton X-100) in DPBS, for 1 hour at normal temperature. For staining collagen type I, a primary antibody (Anti-collagen I polyclonal primary antibody, #PA1-26204) is diluted 1:200 in the blocking solution, and 300 μl of the diluted primary antibody is stored per each cell culture device 100 at 4° C. for 8 to 10 hours. After washing, a secondary antibody (Goat anti-Mouse IgG [H+L] Alexa Fluor™ Plus 488, #A32723) for collagen type I is diluted 1:800 in the blocking solution, and treatment is performed with a staining solution, prepared by diluting Hoechst 33342 for cell nuclei staining 1:2000, at normal temperature for 2 hours. After washing three times for 15 minutes, fluorescence images are captured using a confocal microscope (K1-Fluo, Nanoscope Systems, Korea).

FIG. 6 shows graphs numerically comparing the number of cells (migrated cells) and collagen secretion by culture date in the central region and peripheral regions, (a) of FIG. 6 shows graphs related to the central region where an injury is induced, and (b) of FIG. 6 shows graphs related to the upper right region that is not in contact with the central region among the eight peripheral regions (that is, normal regions where no injury is induced). Referring to (a) of FIG. 6, it can be confirmed that the number of cells in the central region increases over time and that collagen secretion also increases accordingly, and referring to (b) of FIG. 6, it can be seen that the number of cells and collagen secretion in the upper right region change. More specifically, regarding cell proliferation, it can be confirmed that the increased cell count in the injury-induced region illustrated in (a) of FIG. 6 reaches a level comparable to that in the injury non-induced peripheral regions illustrated in (b) of FIG. 6, and regarding the degree of collagen secretion, it can be confirmed that collagen secretion in the injury-induced region illustrated in (a) of FIG. 6 increases by a certain level compared to the amount of collagen secretion in the injury non-induced peripheral regions illustrated in (b) of FIG. 6. Here, cells migrate from the upper, lower, left, and right normal regions directly in contact with the injury-induced central region to the injury-induced central region, resulting in an increase in cell count and collagen secretion in the central region as illustrated in (a) of FIG. 6.

Preferably, the signal intensity of cell nuclei and collagen type I illustrated in FIG. 6 can be quantified through the following process. To analyze the signal intensity of cell nuclei and collagen type I, z-stack images for each cell culture region 111 are acquired using a confocal microscope. A region of interest (ROI) for analysis is set to an x-y axis range of 700 μm×700 μm and a z-axis range of 175 μm based on the center point of the cell culture region 111 so that a frame of the cell culture region 111 is not included in the ROI. An individual ROI is analyzed using Fiji software. Specifically, overlapping nuclei are separated using the Distance Transform Watershed 3D function of Fiji software, and the number of objects is obtained using the 3D manager function to estimate the number of cell nuclei per individual ROI. Collagen type I is calculated as the sum of the volumes of portions showing fluorescence per individual ROI using the 3D volume function of Fiji software.

That is, referring to FIGS. 3 to 6, it can be seen that cell migration, cell proliferation, and collagen secretion necessary for extracellular matrix (ECM) remodeling and tissue stabilization can be smoothly carried out during an injury healing process through an interaction between the through-type cell culture regions 111, and a process in which, after the skin is injured, blood vessels stop bleeding and skin cells are filled with collagen to restore the skin can be reproduced when the cell culture device 100 according to the present invention is used.

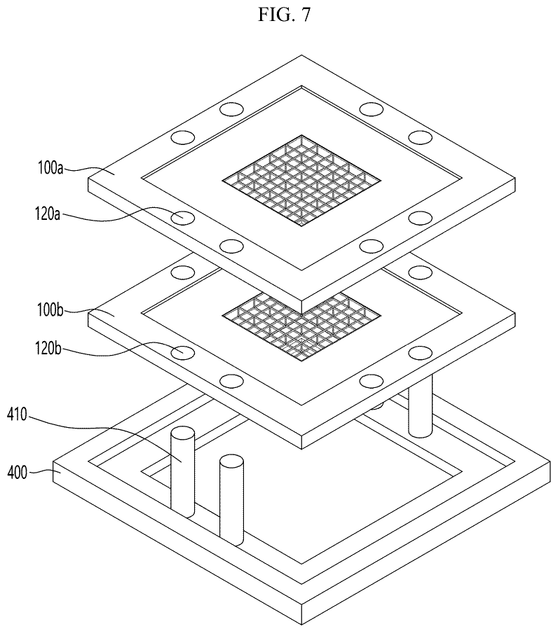

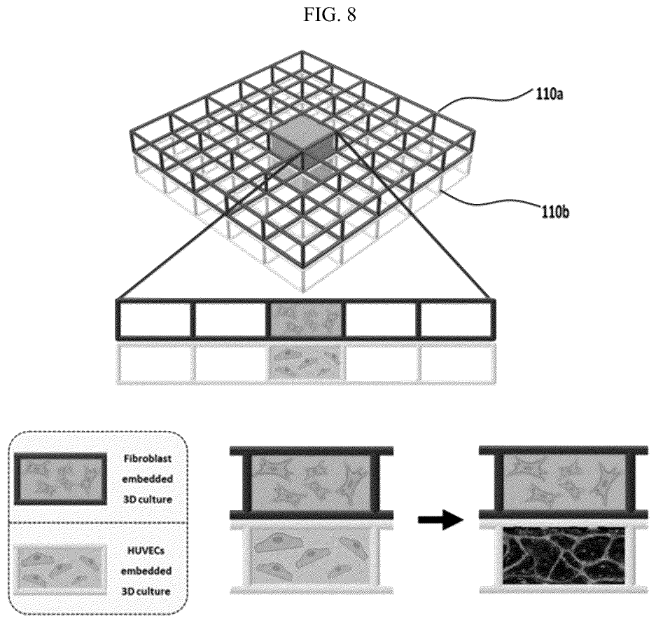

FIG. 7 is a view showing vertical coupling of a plurality of cell culture devices according to one embodiment.

Referring to FIG. 7, a plurality of cell culture devices 100a and 100b include a plurality of through-holes 120a and 120b, respectively, in a region extending from the culture structure portion 110, and the plurality of cell culture devices 100a and 100b may be coupled (that is, stacked) in a vertical direction by cylindrical structure portions 410 provided on a separate holder 400 passing through the plurality of through-holes 120a and 120b.

Preferably, the cell culture devices 100a and 100b may each include a region extending in a plate shape from the culture structure portion 110, and the plurality of through-holes 120a and 120b may be formed in the extending regions. Although it is illustrated in FIG. 7 that the extending regions are each formed in a quadrangular shape, and two through-holes 120a and two through-holes 120b are formed in each of four sides of one of the quadrangular shapes, the shape of the extending regions may be changed in various ways, and the positions where the through-holes 120a and 120b are formed, the number of through-holes 120a and 120b, and the shape of the through-holes 120a and 120b may be changed in various ways as long as the through-holes 120a and 120b are formed to correspond between the plurality of cell culture devices 100a and 100b that are vertically coupled, and movement of the cell culture devices 100a and 100b does not occur after coupling.

Preferably, the holder 400 may include a plurality of cylindrical structure portions 410 that pass through the through-holes 120a and 120b according to the positions, number, and shape of the through-holes 120a and 120b and mechanically couple the plurality of cell culture devices 100a and 100b that are stacked. Here, the shape, structure, and size of the holder 400 may be changed in various ways, and the number of cell culture devices 100 stacked on the holder 400 may be changed in various ways according to the height of the cylindrical structure portions 410, the thickness of each cell culture device 100, or the purpose of stacking the cell culture devices 100. In addition, the holder 400 may be manufactured using stereolithography (SLA) 3D printing, for example, may be manufactured by being printed using black resin V4.1 and then undergoing a post-processing process that corresponds to 10 minutes of washing and 30 minutes of UV curing at 60° C. Here, the type of resin used during 3D printing and the post-processing process may be changed in various ways.

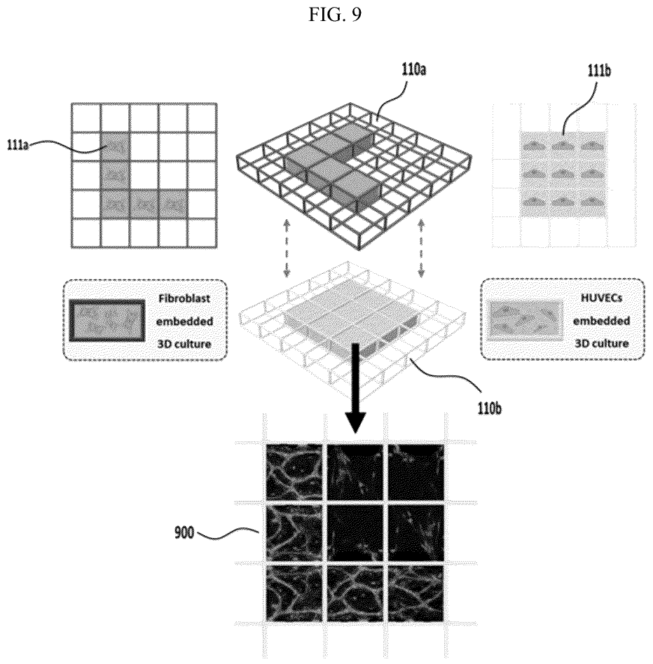

In one embodiment, an interaction between cells may occur due to contact between culture media of corresponding cell culture regions 111 of the plurality of cell culture devices 100a and 100b that are vertically coupled through the holder 400. An interaction between cell culture regions due to vertical coupling of the plurality of cell culture devices 100a and 100b will be described in more detail below with reference to FIGS. 8 and 9.

Referring to FIG. 8 which is an exemplary view showing a case in which a vascularized artificial tissue is formed by stacking two cell culture devices 100a and 100b, in a culture structure portion 110b of the cell culture device 100b located below, human umbilical vein endothelial cells (HUVECs) are patterned with a liquid-state fibrin gel suspension, and in a culture structure portion 110a of the cell culture device 100a located above, human lung fibroblasts (LFs) are patterned with a liquid-state fibrin gel suspension, and then immediately gelled to perform three-dimensional cell culture. Here, LFs secrete a vascular endothelial growth factor (VEGF), and the VEGF induces vascularization of HUVECs. By co-culturing the two cells through the two vertically coupled cell culture devices 100a and 100b, vascularization of artificial tissue by the HUVEC of the cell culture device 100b located below is induced due to the VEGF secreted from the LF of the cell culture device 100a located above.

Preferably, the HUVECs are suspended in a fibrinogen solution and prepared with a final concentration of 5.0×10⁶ cells/ml, and the LFs are suspended in a fibrinogen solution and prepared with a final solution of 5.0×10⁶ cells/ml. Thrombin is added to each suspension at a volume ratio of 50:1, the resulting suspension is patterned at 1.2 μl per cell culture region 111, gelation is induced at normal temperature for 5 minutes, and then the cell culture device 100b in which the HUVECs are patterned and the cell culture device 100a in which the LFs are patterned are sequentially stacked on the holder 400 filled with an endothelial growth medium-2 (EGM-2), and cell culture is performed. A media change is carried out daily, and through a one-week co-culture period, the VEGF is transferred from the cell culture device 100a in which the LFs are patterned to the cell culture device 100a in which the HUVECs are patterned, and vascularization is induced.

Here, proliferation of the HUVECs and LFs may be performed through the following process, and contents related thereto may identically apply to contents described with reference to FIGS. 7 to 15. The HUVECs are cultured using the EGM-2, and a passage number of 4 to 7 is used. The LFs are cultured using a fibroblast growth medium-2 (FGM-2), and a passage number of 5 to 8 is used. A media change is carried out daily for the HUVECs and LFs. The cells are cultured in an incubator in which a temperature is maintained at 37° C. and to which 5% CO₂ is supplied and are used when cell confluency reaches about 80%. For subculture, the HUVECs are treated with 0.05% trypsin-EDTA which is an enzyme mixture and the LFs are treated with 0.25% trypsin-EDTA which is an enzyme mixture to detach the cells, and the detached cells are centrifuged at 169×g for 3 minutes and resuspended in the corresponding cell culture medium.

Referring to FIG. 9 which shows a case in which the LFs are patterned only in specific cell culture regions 111 in the culture structure portion 110a located above, referring to the figure denoted by reference numeral 900, it can be confirmed that vascularization of an artificial tissue is induced in cell culture regions 111b that correspond to cell culture regions 111a in which the LFs are patterned in the culture structure portion 110a located above among the cell culture regions 111b in which the HUVECs are patterned in the culture structure portion 110b located below. This shows that it is possible to realize an interaction between different cells in the two vertically coupled cell culture devices 100a and 100b.

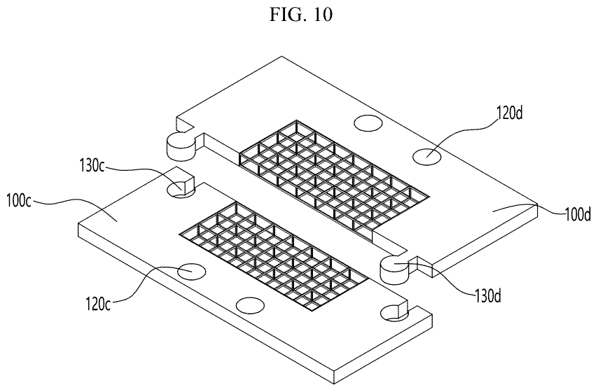

FIG. 10 is a view showing horizontal coupling of a plurality of cell culture devices according to one embodiment.

Referring to FIG. 10, a plurality of cell culture devices 100c and 100d each include a plurality of engraved or embossed structure portions 130c or 130d formed on corresponding one surface in a region extending from the culture structure portion 110. Preferably, the engraved structure portions 130c may be formed in one cell culture device 100c of the plurality of cell culture devices 100c and 100d, the embossed structure portions 130d may be formed on the other cell culture device 100d, and the embossed structure portions 130c and the engraved structure portions 130d may be formed at corresponding positions to enable horizontal coupling between the cell culture devices 100c and 100d.

In one embodiment, during horizontal coupling through the engraved structure portions 130c and the embossed structure portions 130d, direct contact may occur between culture structure portions 110c and 100d on surfaces in contact between the plurality of cell culture devices 100c and 100d, and in this way, an interaction between cells may occur due to contact between culture media of the cell culture regions 111 located at coupling surfaces of the plurality of cell culture devices 100c and 100d. The shapes of the engraved structure portions 130c and the embossed structure portions 130d, the positions at which the engraved structure portions 130c and the embossed structure portions 130d are formed, or the number of the engraved structure portions 130c and the embossed structure portions 130d may be changed in various ways as long as an interaction may occur at the coupling surfaces of the plurality of cell culture devices 100c and 100d.

In addition, although it is illustrated in FIG. 10 that two cell culture devices 100c and 100d are horizontally coupled through the engraved structure portions 130c and the embossed structure portions 130d, the number of horizontally coupled cell culture devices may be changed in various ways, and accordingly, the number of engraved or embossed structure portions formed on one cell culture device and the positions of the engraved or embossed structure portions may also be changed in various ways. For example, a plurality of cell culture devices 100 having engraved or embossed structure portions formed on two surfaces facing each other may be horizontally coupled in series, or a plurality of cell culture devices 100 having embossed or engraved structure portions formed on two continuing surfaces may be horizontally coupled in the x-axis direction or the y-axis direction.

Preferably, the plurality of cell culture devices 100c and 100d may include a plurality of through-holes 120c and 120d described with reference to FIG. 7, in addition to including the engraved structure portions 130c or the embossed structure portions 130d. The plurality of horizontally coupled cell culture devices 100c and 100d may be fixed by cylindrical structure portions 410 provided on a separate holder 400 passing through the plurality of through-holes 120c and 120d. Here, because contents described above with reference to FIG. 7 may identically apply to configurations of the plurality of through-holes 120c and 120d, detailed description thereof will be omitted herein.

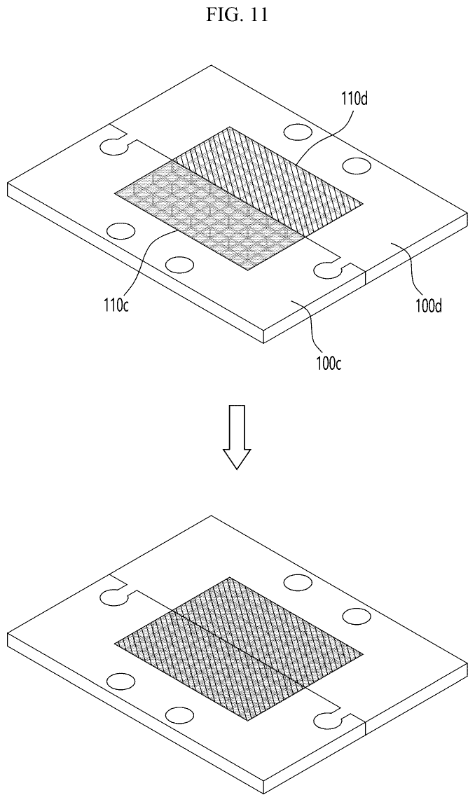

In one embodiment, referring to FIG. 11 which is an exemplary view showing a case in which an interaction occurs between two cell culture devices 100c and 100d according to horizontal coupling, it is shown that a cell-free hydrogel suspension having a pigment added thereto is patterned and gelled in each of the two cell culture devices 100c and 100d, and the engraved structure portions 130c and the embossed structure portions 130d are connected to horizontally couple the two cell culture devices 100c and 100d. The culture structure portions 110c and 110d of the two cell culture devices 100c and 100d may have different colors, but the colors may ultimately be mixed through horizontal coupling, and this means that an interaction such as material exchange occurs after horizontal coupling between the two cell culture devices 100c and 100d. Different colors are indicated by dots and hatching in FIG. 11, and for example, in a case where the culture structure portion 110c indicated by dots is in a state in which a suspension having a blue pigment added thereto is patterned and gelled, and the culture structure portion 110d indicated by hatching is in a state in which a suspension having a red pigment added thereto is patterned and gelled, according to the occurrence of an interaction due to horizontal coupling between the two cell culture devices 100c and 100d, colors of the two culture structure portions 110c and 110d may gradually change to a color that is a mixture of red and blue, starting from the coupling surfaces.

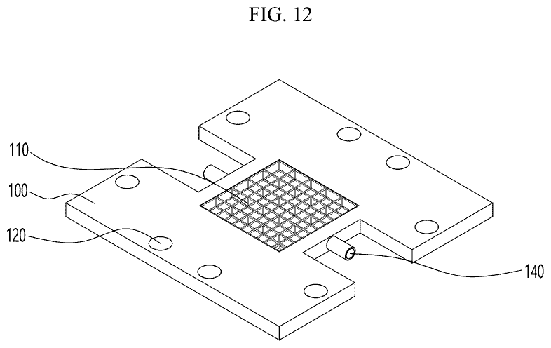

Preferably, horizontal coupling between the plurality of cell culture devices 100c and 100d may be used when differences occur in preparation and stabilization periods depending on the cell type in experiments using co-culture of different cells. For example, in a case where spheroids are cultured in one cell culture device 100c, and perfusable vascular structures made of HUVECs are cultured in the other cell culture device 100d (which will be described in detail below with reference to FIG. 12), after a series of processes necessary for device preparation, such as patterning, gelation, or microneedle insertion, is performed in the cell culture device 100d, the two cell culture devices 100c and 100d are coupled to allow subsequent experiments to be performed continuously. That is, experimental convenience can be provided because an experimenter can independently prepare an experiment and culture cells in each cell culture device and then continue cell culture by horizontally coupling the plurality of cell culture devices 100c and 100d at an optimal point in time.

FIG. 12 is a view of a cell culture device including a cylindrical portion according to one embodiment.

Referring to FIG. 12, a cell culture device 100 includes a culture structure portion 110, through-holes 120, and cylindrical portions 140. The culture structure portion 110 and the through-holes 120 have been described in detail above and thus will not be described here.

The cylindrical portions 140 are formed at both ends of the culture structure portion 110 to enable liquid perfusion into a series of cell culture regions 111 among the plurality of cell culture regions 111. That is, the cylindrical portions 140 are formed in a structure in which a microneedle that has passed via the cylindrical portion 140 formed at one end is able to pass through the series of cell culture regions 111 and then exit through the cylindrical portion 140 formed at the other end. Although the cylindrical portions 140 are formed as a pair to correspond to the cell culture regions 111 located in the middle row among the plurality of cell culture regions 111 in FIG. 12, the positions where the cylindrical portions 140 are formed and the number of cylindrical portions 140 may be changed in various ways. In addition, although not illustrated in FIG. 12, the cell culture device 100 may be formed to include the engraved or embossed structure portion described above with reference to FIG. 10. At this time, conditions in which the engraved or embossed structure portion is formed may be the same as those described above.

In one embodiment, a vascular structure may be formed through the cylindrical portions 140 of the cell culture device 100. A hydrogel of a state is patterned in the cell culture device 100, a microneedle is passed via the cylindrical portions 140 prior to gelation, and gelation is induced in that state. That is, gelation is carried out in a state in which the microneedle is inserted into the series of the plurality of cell culture regions 111 via the cylindrical portions 140. When the microneedle is removed after the gelation, a hollow lumen shape may be formed in the patterned hydrogel in the cell culture device 100. In addition, liquid perfusion is possible through the formed lumen when tubing is connected to the cylindrical portions 140 and a syringe pump is used.

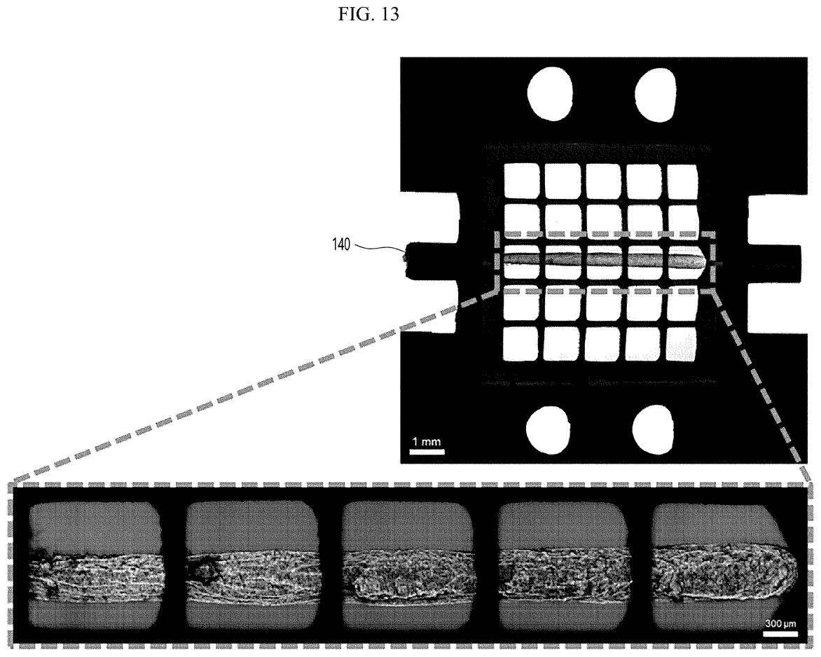

Preferably, gelation is induced for 30 minutes in an incubator in which a temperature is maintained at 37° C. and to which 5% CO₂ is supplied, while 2.5 mg/ml of cell-free collagen type I gel is patterned at 1.2 μl per cell culture region 111, and a microneedle is inserted into the cylindrical portions 140. A lumen is formed when the inserted microneedle is removed after gelation, and as illustrated in FIG. 13, after tubing and a syringe pump are connected to the cylindrical portions 140, and fluorescent particles (Green PS fluorescent particles, 500 nm, #ABWG- 21-0050) are injected into the lumen formed in the cell culture device 100, the cell culture device 100 is held in a holder filled with DPBS, and then stored and imaged. That is, referring to FIG. 13 which shows an example in which tubing and a syringe pump are connected to the cylindrical portions 140 and fluorescent particles are injected into the cell culture device 100, it is confirmed that the fluorescent particles are injected into the formed lumen.

In another embodiment, in a case where spheroids are cultured in a cell culture device 100e in which a lumen is formed, 2.5 mg/ml of cell-free collagen type I gel is patterned at 1.2 μl per cell culture region 111, a microneedle is inserted into the cylindrical portions 140, and then one spheroid per cell culture region 111 is loaded together with collagen type I gel. Then, gelation is induced for 30 minutes in an incubator in which a temperature is maintained at 37° C. and to which 5% CO₂ is supplied, the inserted microneedle is removed, the cell culture device 100e is held in a structure filled with D10, and cell culture is performed for 3 days. A media change is carried out daily.

By injecting a HUVEC suspension into the lumen formed as above, a lumen coated with HUVEC is formed and can serve to transport materials through liquids like actual blood vessels, and the cell culture device 100 that has a vascular structure formed therein, i.e., that is vascularized, can form a scaled-up artificial tissue.

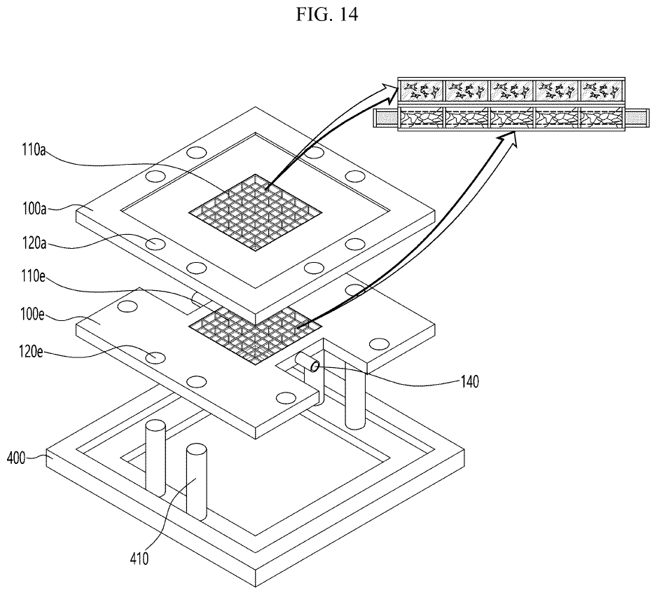

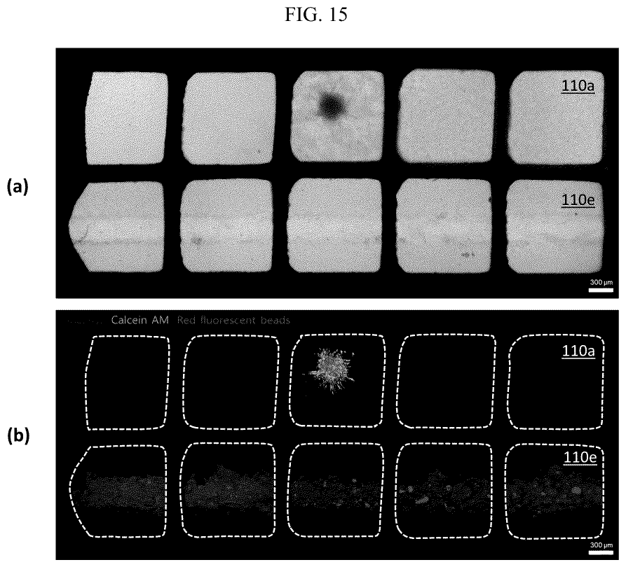

FIGS. 14 and 15 are views for describing an interaction between cell culture regions using the vascular structure formed in the cell culture device including the cylindrical portions according to one embodiment.

An experiment for growing capillaries above and below a vascular structure can be conducted using the cell culture device 100 in which the vascular structure described above with reference to FIGS. 12 and 13 is formed. Referring to FIG. 14, the cell culture device 100e, in which a vascular structure is formed through the cylindrical portions 140, is vertically coupled to another cell culture device 100a through a holder, and culture of spheroids or organoids is possible through an interaction between the plurality of cell culture devices 100a and 100e that are vertically coupled. In addition, capillaries may grow from the blood vessels formed through the cylindrical portions 140 in a culture structure portion 110e of the same cell culture device 100e, or capillaries may grow from the blood vessels formed through the cylindrical portions 140 or the capillaries of the culture structure portion 110e in a culture structure portion 110a of the other cell culture device 100a, that is, the cell culture device 100a stacked on the cell culture device 100e.

Preferably, a lumen may be formed in the cell culture device 100e, 3T3 fibroblast spheroids may be patterned in a specific cell culture region 111 in the cell culture device 100a, the cell culture devices 100a and 100e may be vertically coupled, and cell culture may be performed in both the cell culture devices 100a and 100e. Here, the spheroid or organoid is a spherical body in which cells are three-dimensionally aggregated and is a structure that can partially mimic the characteristics of tissues in the body. In the cell culture device 100a, various types of spheroids or organoids can be precisely positioned in a specific cell culture region 111 while mixed with a hydrogel and can be cultured for a long period of time to reproduce a three-dimensional structure.

Preferably, because an interaction is possible between the plurality of cell culture devices 100a and 100e that are vertically coupled, a nutrient or a specific drug perfused through the lumen formed in the cell culture device 100e can reach a central portion of the spheroid or organoid patterned in the cell culture device 100a, and a survival rate of the spheroid or organoid may increase or decrease. For example, when an anti-cancer drug is perfused through the lumen formed in the cell culture device 100e, a survival rate of a cancer spheroid of the cell culture device 100a may decrease. Referring to FIG. 15 which is an exemplary view showing a case where the cell culture device 100e having a lumen formed therein and the cell culture device 100a in which a spheroid or an organoid is patterned at a specific position are vertically coupled, whether a survival rate of a spheroid or an organoid patterned in a specific cell culture region 110a of the cell culture device 100a increases, stays the same, or decreases can be experimented using a nutrient or a specific drug perfused in the cell culture region 110e through co-culture of spheroids and blood vessels. This shows that the cell culture device 100 according to the present invention can be utilized in evaluation of drug response through perfusion, modulation of culture environment, or modeling of blood flow-based diseases such as cancer metastasis.

Preferably, 3T3 fibroblast spheroids patterned in the cell culture device 100a may be prepared according to the following process. ¼ of a 500 μm SpheroFilm, which is a microstructure plate for spheroid formation, is attached to a 60-mm diameter culture dish (60 pi petri dish) and is irradiated with UV for 30 minutes, 3 ml of 3T3 fibroblast suspension with a concentration of 10.0×10⁶ cells/ml is added thereto, cell culture is performed in an incubator, in which a temperature is maintained at 37° C. and to which 5% CO₂ is supplied, using D10 (a complete medium in which 10% fetal bovine serum (FBS) is added to Dulbecco's Modified Eagle Medium (DMEM)), and a media change is carried out daily. Cell culture is performed for 8 days under the above conditions so that the size of spheroids is about 300 μm.

The description of the presented embodiments is provided so that those of ordinary skill in the art to which the present invention pertains are able to use or carry out the present invention. Various modifications to the embodiments will be apparent to those of ordinary skilled in the art to which the present invention pertains, and generic principles defined herein may be applied to other embodiments without departing from the scope of the present invention. Therefore, the present invention is not limited to the embodiments presented herein, and the present invention should be construed within the widest range which is coherent with the principles and novel features presented herein.

### DESCRIPTION OF REFERENCE NUMERALS

100: cell culture device
110: culture structure portion
111: cell culture region
120: through-hole
130: engraved or embossed structure portions
140: cylindrical portions
200: culture medium
300: pipette
400: holder
410: cylindrical structure portion

1. A cell culture device comprising:
a culture structure portion including a plurality of cell culture regions,
wherein, in the culture structure portion, a culture medium for culturing a cell is maintained in each of the plurality of cell culture regions due to surface tension, and the plurality of cell culture regions are formed with a through-type structure so that an interaction is possible between adjacent cell culture regions among the plurality of cell culture regions.

2. The cell culture device of claim 1, wherein the plurality of cell culture regions are each formed with a through-type cube structure of a predetermined size, and a plurality of different cells are simultaneously cultured through different culture media located in the through-type cube structures.

3. The cell culture device of claim 2, wherein, based on an inner space of the through-type cube structure, the through-type cube structure has a size in which each of a width, a length, and a height ranges from 0.35 mm to 2 mm.

4. The cell culture device of claim 1, wherein:
the culture structure portion further includes a coupling portion mechanically coupled to another cell culture device; and
an interaction is possible between cell culture regions of a plurality of cell culture devices coupled through the coupling portion.

5. The cell culture device of claim 4, wherein:
the coupling portion includes a plurality of through-holes to enable vertical coupling with the other cell culture device through a holder having a specific structure; and
an interaction between cells is possible due to contact between culture media of corresponding cell culture regions of the vertically-coupled plurality of cell culture devices.

6. The cell culture device of claim 4, wherein:
the coupling portion includes an embossed or engraved structure portion to enable horizontal coupling with the other cell culture device; and
an interaction between cells is possible due to contact between culture media of cell culture regions located on coupling surfaces of the horizontally-coupled plurality of cell culture devices.

7. The cell culture device of claim 1, wherein the culture structure portion further includes cylindrical portions formed at both ends of the plurality of cell culture regions to enable liquid perfusion to a series of cell culture regions among the plurality of cell culture regions.

8. A multi-region liquid patterning method using a cell culture device including a culture structure portion including a plurality of cell culture regions, the multi-region liquid patterning method comprising:
patterning a liquid inside each of the plurality of cell culture regions by passing a pipette containing a culture medium for cell culture through each of the plurality of cell culture regions and then, when lifting the pipette, using surface tension generated between the culture medium formed on a tip of the pipette and the cell culture regions formed with a through-type structure,
wherein an interaction occurs between adjacent cell culture regions among the plurality of cell culture regions.

9. The multi-region liquid patterning method of claim 8, wherein the patterning of the liquid includes:
patterning a culture medium including human vascular endothelial cells inside each of the plurality of cell culture regions of the cell culture device;
patterning a culture medium including human lung fibroblasts inside each of a plurality of cell culture regions of another cell culture device that is able to be vertically coupled to the cell culture device;
mechanically coupling the cell culture device and the other cell culture device through a coupling portion formed in a culture structure portion so that the cell culture device is located below and the other cell culture device is located above; and
forming a vascularized artificial tissue in the cell culture device located below according to an interaction between cells that occurs due to contact between culture media of corresponding cell culture regions of the cell culture device and the other cell culture device.

10. The multi-region liquid patterning method of claim 8, wherein the patterning of the liquid includes:
patterning a sol-state hydrogel inside a series of cell culture regions having cylindrical portions formed at both ends among the plurality of cell culture regions;
passing a microneedle through the series of cell culture regions via the cylindrical portions and inducing gelation; and
when the microneedle is removed after the gelation, forming a vascular structure in which liquid perfusion is possible.
